(19) United States
(12) Patent Application Publication
(10) Pub. No.: US 2026/0200855 A1
(43) Pub. Date: Jul. 16, 2026

(54) VANILLIN PYRIDINE CARBOXYLATES

(71) Applicant: Renovel Innovations, Inc., Fremont, CA (US)

(72) Inventors: Bishwajit NAG, Union City, CA (US); Ananda Sen, Castro Valley, CA (US); Nitish Nag, Union City, CA (US); Srinivasan Narasimahan, Chennal (IN)

(21) Appl. No.: 19/448,773

(22) Filed: Jan. 14, 2026

Related U.S. Application Data
(60) Provisional application No. 63/745,166, filed on Jan. 14, 2025.

(51) Int. Cl. C07D 213/80; A61K 31/455; C07D 213/807

(52) U.S. Cl. CPC C07D 213/80; A61K 31/455; C07D 213/807

(57) ABSTRACT

Novel Vanillin Pyridine Carboxylates are provided which exhibit activity for the treatment of immunological diseases, inflammation, obesity, hyperlipidemia, hypertension, neurological diseases and diabetes, and for reducing plasma glucose, free fatty acids, cholesterol, and triglyceride levels, and for treating obesity, autoimmune diseases, inflammation, immunological disease, and disorders associated with insulin resistance.

### FIELD OF THE INVENTION

The present invention relates to novel styryl carboxylates for the treatment of immunological diseases, inflammation, obesity, hyperlipidemia, hypertension, neurological diseases and diabetes.

### BACKGROUND OF INVENTION

Metabolic syndrome, Insulin resistance syndrome or Syndrome X is a name for a group of risk factors that occur together and increase the risk for coronary artery disease, stroke, and type 2 diabetes. Metabolic syndrome is becoming more and more common globally specially in the United States. Researchers are not sure whether the syndrome is due to one single cause, but all of the risks for the syndrome are related to obesity. The two most important risk factors for metabolic syndrome are: Extra weight around the middle and upper parts of the body (central obesity) and insulin resistance. The body uses insulin less effectively than normal. Insulin is needed to help control the amount of sugar in the body. As a result, blood sugar and fat levels rise. Other risk factors include Aging, Genes, Hormone changes, Lack of exercise. People who have metabolic syndrome often have two other problems that can either cause the condition or make it worse. Excess blood clotting, and increased levels of blood substances that are a sign of inflammation throughout the body.

Metabolic syndrome is affiliated with three or more of the following signs: Blood pressure equal to or higher than 130/85 mmHg, Fasting blood sugar (glucose) equal to or higher than 100 mg/dL, Large waist circumference (length around the waist Men-40 inches or more and Women-35 inches or more, Low HDL cholesterol (Men-under 40 mg/dL Women-under 50 mg/dL) and Triglycerides equal to or higher than 150 mg/dL. In general, metabolic syndrome is a combination of Type 2 Diabetes, Obesity, Hyperlipidemia and hypertension.

People with metabolic syndrome have an increased long-term risk for developing heart disease, type 2 diabetes, stroke, kidney disease, and poor blood supply to the legs. There is no one single treatment option available to treat metabolic syndrome. Current drugs that control blood glucose are usually not effective in lowering body weight, hypertension and cholesterol. Similarly, drugs that manage lipid levels may or may not have impact on other metabolic parameters. The present invention was aimed to develop new class of therapeutics derived, modified and chemically synthesized from natural product which can combat multiple arms of metabolic syndrome. The invention also describes one such core group of molecules with synthesis scheme and biological data for diabetes, obesity, inflammation, hypertension and Hyperlipidemia.

### SUMMARY OF INVENTION

The present invention relates to novel Vanillin Pyridine Carboxylates of the formula I (see FIG. 1), their derivatives, their analogs, their tautomeric forms, their stereoisomers, their polymorphs, their pharmaceutically acceptable salts, their pharmaceutically acceptable solvates, wherein the pyridine moiety is either unsubstituted (A=B=C=E=Hydrogen) or substituted with hydroxyl, alkoxy or halogen groups at suitable positions and the carboxylic group present at the meta position of the hetero nitrogen atom is esterified with the hydroxyl functional group of vanillin moiety and also the aldehyde group of the vanillin moiety is either free or forms a condensation analogue with the free amino group present in any synthetic or naturally occurring amino acid, its corresponding ester or alcohol derivatives, or an hydroxylamine or substituted aliphatic or aromatic hydrazines. R3 is always a methyl group and R1 and R2 can be selected from various groups.

The present invention also relates to a process for the preparation of the above said novel compounds, their analogues, their tautomeric forms, their stereoisomers, their polymorphs, their pharmaceutically acceptable salts, their pharmaceutically acceptable solvates, novel intermediates and pharmaceutical composites containing them. Tautomeric forms are isomeric forms which exists in a state of equilibrium capable of reacting according to either form. Stereoisomers include configurational isomers, such as cis- and trans double bonds, as well as optically active isomers having different spatial arrangements of their atoms. Polymorphs are molecules which can crystallize in two or more forms. Solvates are molecular or ionic complexes of molecules or ions of solvent with those of a solute. The amino acid derivatives are included, but not limited to naturally occurring amino acids. Analogues include those compounds which differ by substitution of an oxygen, sulphur, nitrogen or carbon atom in place of such an atom. Analogues also include atoms of the same family of the Periodic Table, such as F, Cl, Br and I. Derivatives include compounds resulting from routine functionalizing of atoms, such as, derivatives found by protecting amino or carboxyl groups by carboxylation or esterification, respectively.

### BRIEF DESCRIPTION OF FIGURES

FIG. 1 shows the formula I with the general structure used in the patent.

FIG. 2 depicts the process for the preparation of the compounds represented by formula I.

FIG. 3 shows the various R1, R2, R3 representations and IUPAC names for each of the compounds.

FIG. 4 shows treatment with Compound 1 (RNV-133) reduced the high fat diet induced increase in body weight in C57BL/6 mice by 9%.

FIG. 5 shows the effect of Compound 1 (RNV-133) on fasting blood glucose after 30 days of treatment in C57BL/6 mice fed on a high fat diet

FIG. 6 shows that Compound 1 (RNV-133) reduced total cholesterol by 5% and triglyceride by 30% in C57BL/6 mice fed on a high fat diet.

FIG. 7 shows that Compound 1 (RNV-133) significantly (P<0.03) reduced the body weight by 51% in db/db mice after 21 days of treatment.

FIG. 8 shows the time-course effect of Compound 1 (RNV-133) on fasting blood glucose in db/db mice (P<0.0001)

FIG. 9 shows that Compound 1 (RNV-133) significantly (P<0.001) reduced the fasting blood glucose in db/db mice after 21 days of treatment.

FIG. 10 shows that Compound 1 (RNV-133) in an oral glucose tolerance test reduced the blood glucose by 47% in db/db mice.

FIG. 11 shows the effect of Compound 1 (RNV-133) on body weight in Syrian Golden Hamsters fed on a high fructose diet after 61 days of treatment.

FIG. 12 shows the effect of Compound 1 (RNV-133) on total cholesterol in Syrian Golden Hamsters fed on a high fructose diet after 61 days of treatment.

FIG. 13 shows the effect of Compound 1 (RNV-133) on fasting blood glucose in Syrian Golden Hamsters fed on a high fructose diet after 61 days of treatment.

FIG. 14 shows that treatment with Compound 1 (RNV-133) for 61 days significantly (P<0.001) reduced total triglycerides by 47% in Syrian Golden Hamsters fed on a high fructose diet for 61 days.

FIG. 15 shows the effect of Compound 1 (RNV-133) on HDL in Syrian Golden Hamsters fed on a high fructose diet for 61 days.

FIG. 16 shows the effect of Compound 1 (RNV-133) on LDL in Syrian Golden Hamsters fed on a high fructose diet for 61 days.

FIG. 17 shows that treatment with Compound 1 (RNV-133) significantly (P<0.0001) reduced LPS-induced increase in TNF-alpha in mice by 62%

FIG. 18 shows the effect of Compound 2 (RNV-168) on body weight in C57BL/6 mice fed on a high fat diet

FIG. 19 shows that that treatment with Compound 2 (RNV-168) for 60 days reduced the fasting blood glucose in C57BL/6 mice fed on a high fat diet.

FIG. 20 the effect of Compound 3 (RNV-203) on body weight in C57BL/6 mice fed on a high fat diet.

FIG. 21 shows that treatment with Compound 3 (RNV-203) for 60 days significantly (P<0.007) reduced an increase in fasting blood glucose in C57BL/6 mice fed on a high fat diet.

FIG. 22 shows that treatment with Compound 4 (RNV-236) for 90 days significantly (P<0.006) reduced the body weight by 44% in C57BL/6 mice fed on a high fat diet.

FIG. 23 shows that treatment with Compound 4 (RNV-236) for 60 days significantly (P<0.01) decreased the fasting blood glucose in C57BL/6 mice fed on a high fat diet.

FIG. 24 shows that treatment with Compound 4 (RNV-236) decreased the oral glucose tolerance test blood glucose at 30 minutes in C57BL/6 mice fed on a high fat diet.

FIG. 25 shows the time course effect of treatment with Compound 4 (RNV-236) on body weight in C57BL/6 mice fed on a high fat with high sucrose (HFS) diet.

FIG. 26 shows the effect of Compound 4 (RNV-236) on fasting blood glucose in C57BL/6 mice fed on a high fat with high sucrose (HFS) diet.

FIG. 27 shows the effect treatment with Compound 4 (RNV-236) for 56 days on body weight in Syrian Golden Hamsters fed on a fructose diet.

FIG. 28 shows the effect of Compound 4 (RNV-236) on blood glucose in Syrian Golden Hamsters.

FIG. 29 Effect of treatment with Compound 4 (RNV-236) for 56 days on total triglycerides in Syrian Golden Hamsters fed on a fructose fed diet.

FIG. 30 shows that treatment with Compound 4 (RNV-236) for 56 days reduced the total cholesterol in Syrian Golden Hamsters fed on a fructose diet.

### DETAILED DESCRIPTION OF THE INVENTION

In an embodiment of the present invention, the groups represented as A, B, C, E are selected from groups such as Hydrogen, Hydroxyl, Alkoxy, alkyl groups with straight chain or branched and halogens like Chlorine, Fluorine, Bromine and Iodine.

In an embodiment of the present invention, the group represented as R1 can be selected from groups like Hydrogen, Hydroxyl, Alkoxy, alkyl groups with straight chain or branched and halogens like Chlorine, Fluorine, Bromine and Iodine.

In an embodiment of the present invention, the group represented as R3 is always a methyl group.

In an embodiment of the present invention, the group represented as R2 can be selected from amino acids (naturally occurring and synthetic), their corresponding amino acid alcohols and amino acid esters, hydroxylamines and 2,4 or 3,5 di substituted phenyl hydrazines where the substituents can be H, Halogens or Nitro group.

In an embodiment of the present invention, the amino acid group that can be at R2 is selected from alanine, glycine, arginine, aspargine, cysteine, glutamic acid, glutamine, histidine, isoleucine, leucine, lysine, methionine, ornithine, proline, serine, threonine, tryptophan, tyrosine and the like, which may be unsubstituted or substituted and their derivatives such as ester and amides of carboxylic acid. The preferred substituents are selected from halogen, alkyl, alkoxy, aryl, heteroaryl, amino and the like.

Pharmaceutically acceptable salts forming part of this invention include base addition salts such as alkali metal salts like Li, Na, and K salts, alkaline earth metal salts like Ca and Mg salts, salts of organic bases such as lysine, arginine, guanidine, diethanolamine, chlorine and the like, ammonium or substituted ammonium salts. Salts may include acid addition salts which are sulphates, nitrates, phosphates, perchlorates, borates, hydrohalides, acetates, tartarates, maleates, citrates, succinates, palmoates, methanesulphonates, benzoates, ascorbates, glycerophosphates, ketoglutarates and the like. Pharmaceutically acceptable solvates may be hydrates or comprising other solvents of crystallization such as alcohols.

More preferably, the present innovation relates to novel Vanillin Pyridine Carboxylates of formula I (see FIG. 1), their derivatives, their analogues, their tautomeric forms, their stereoisomers, their polymorphs, their pharmaceutically acceptable salts, their pharmaceutically acceptable solvates, novel intermediates and pharmaceutical composites containing them, wherein the pyridine moiety is either unsubstituted (A=B=C=E=Hydrogen) or substituted with hydroxyl, alkoxy or halogen groups at suitable positions and the carboxylic group present at the meta position to the hetero nitrogen atom is esterified with the hydroxyl functional group of vanillin moiety and also the aldehyde group of the vanillin moiety forms a condensation analogue with the free amino group with an alkyl, aryl of any amino acid, or an hydroxylamine or a hydrazine. The group represented as R1 is preferentially selected from groups like Hydrogen, Hydroxyl, Alkoxy, Alkyl groups with straight chain or branched and halogens like Chlorine, Fluorine, Bromine and Iodine. The group represented as R2 is preferentially selected from amino acids (naturally occurring and synthetic), their corresponding amino alcohols and amino esters, hydroxylamines and 2,4 or 3,5 di substituted phenyl hydrazines where the substituents can be H, Halogens or Nitro group. The group represented as R3 is preferentially selected from groups like hydrogen or long/short chain alkyl groups which can be either branched or straight chain.

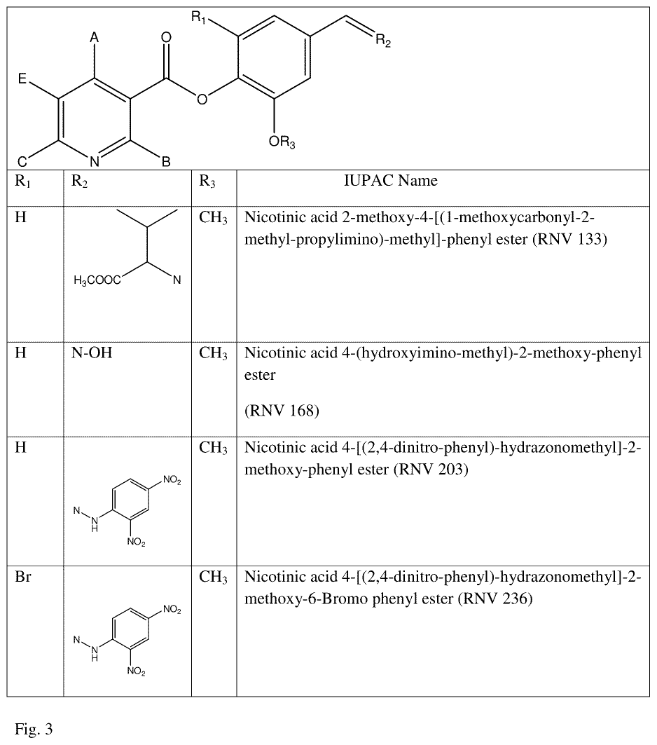

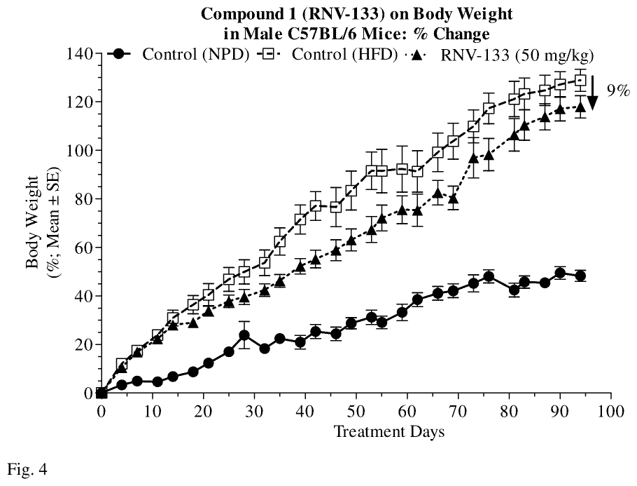

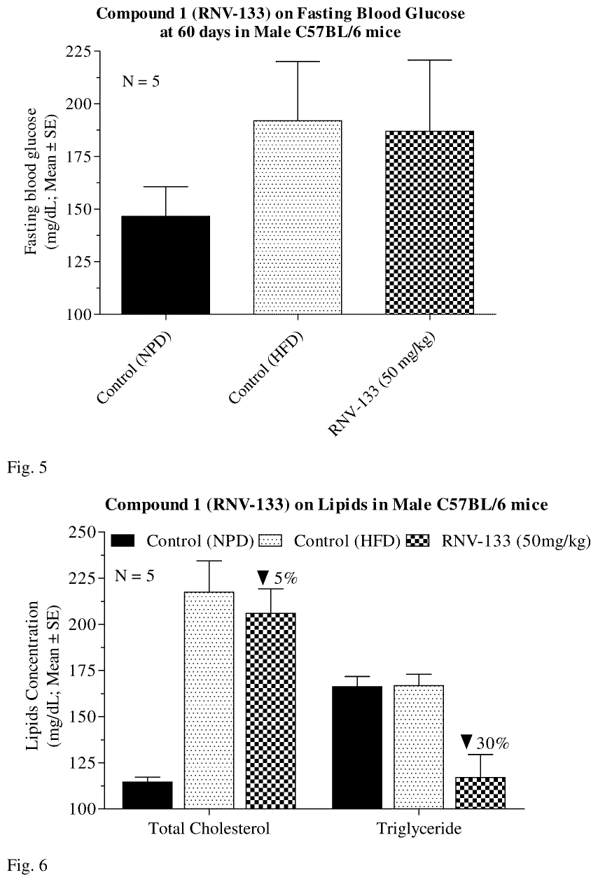

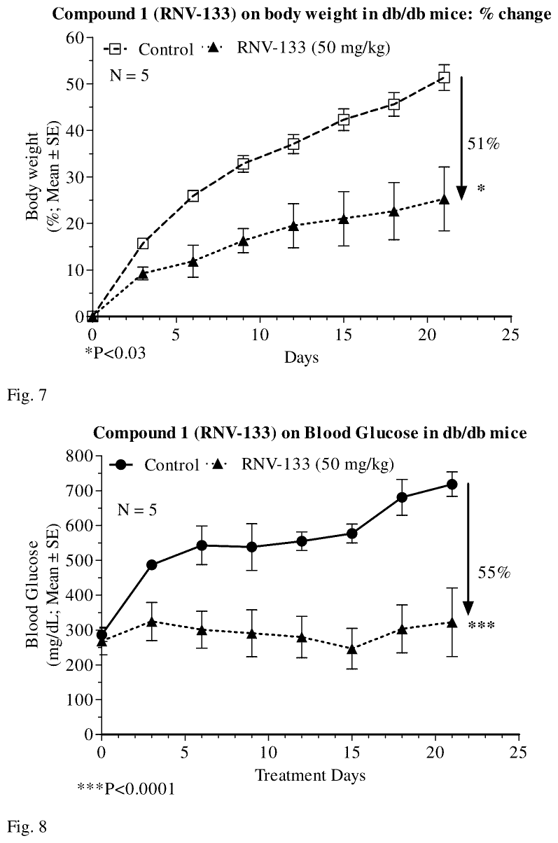

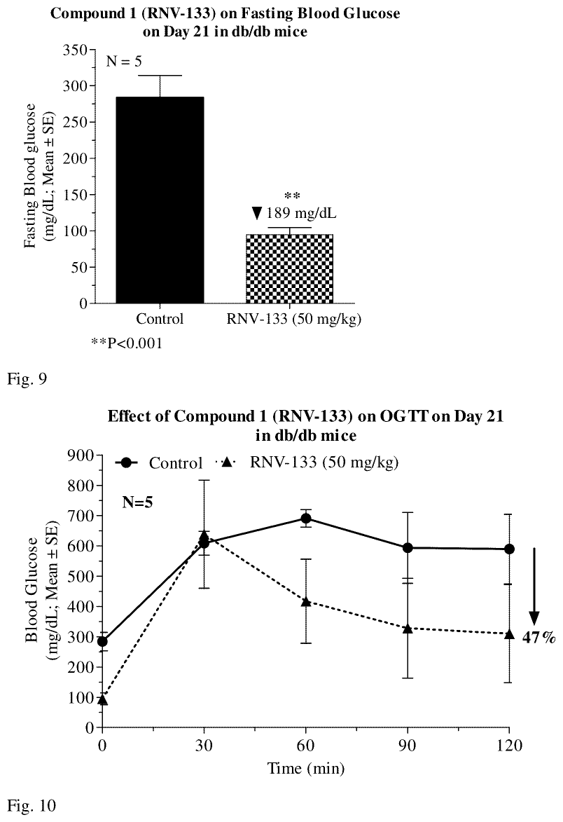

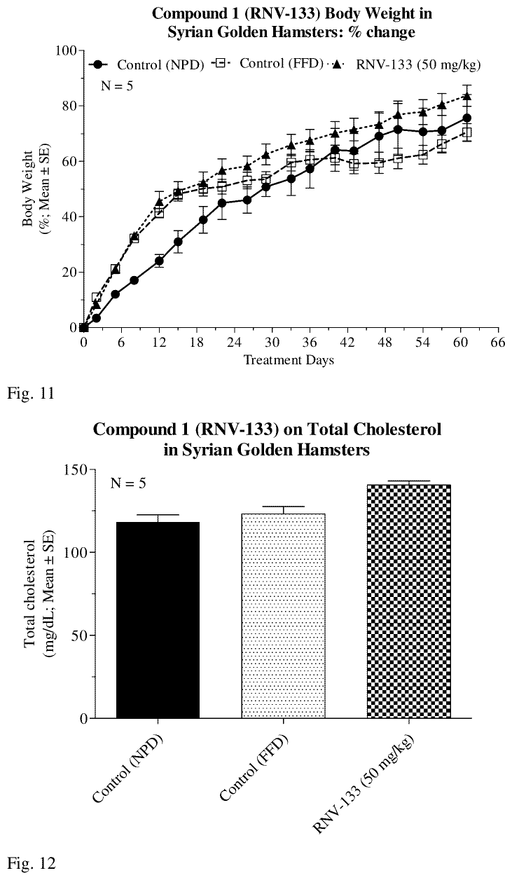

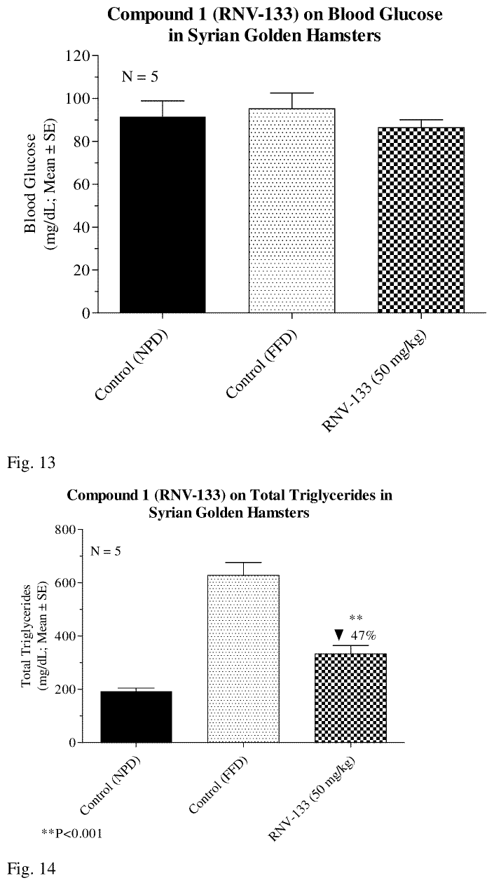

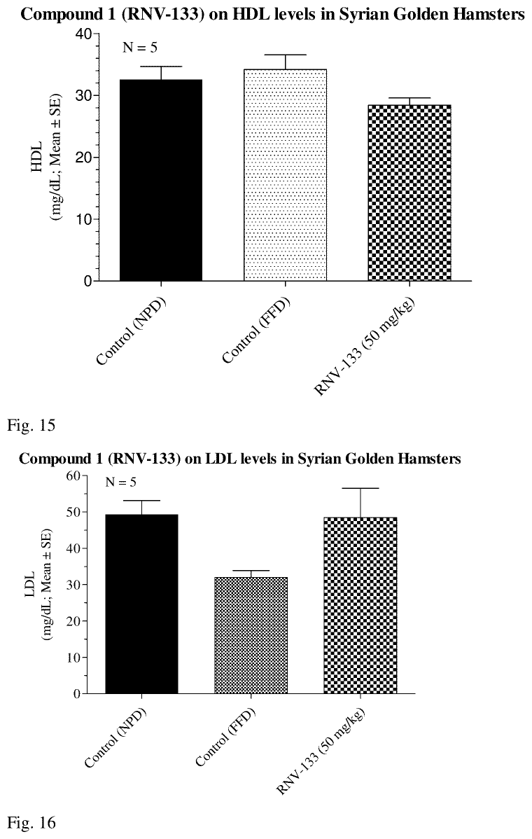

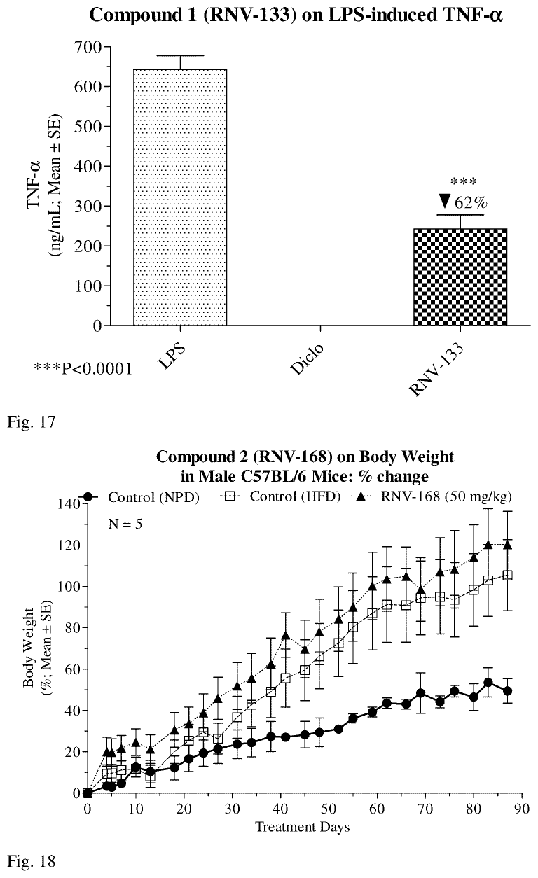

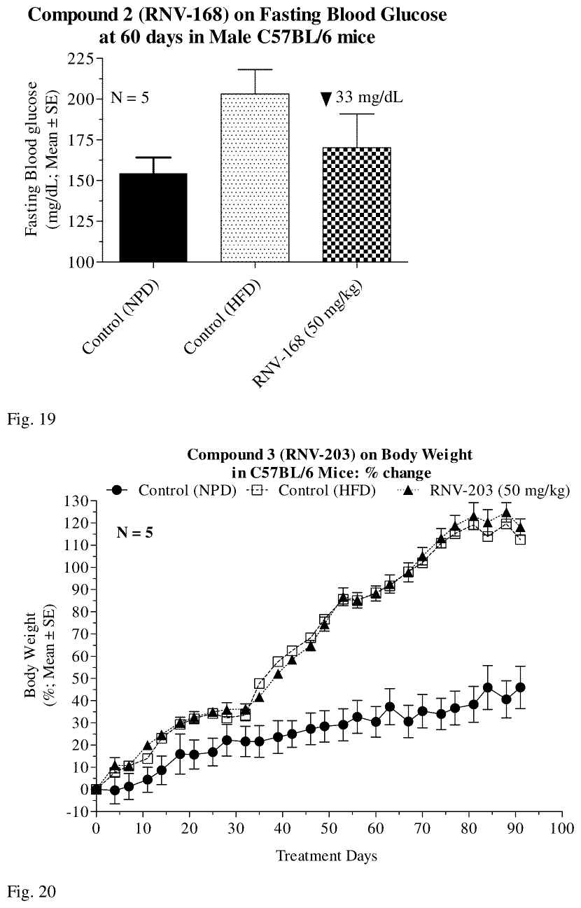

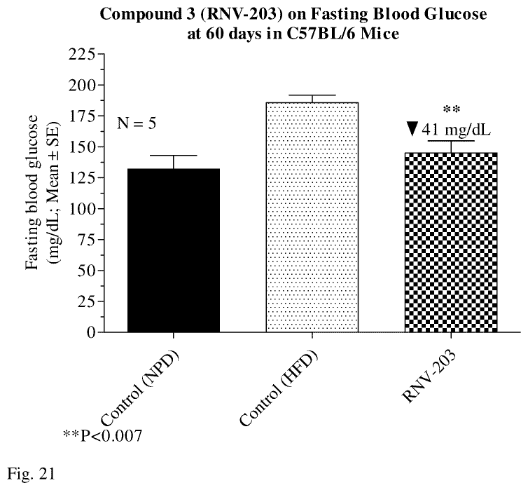

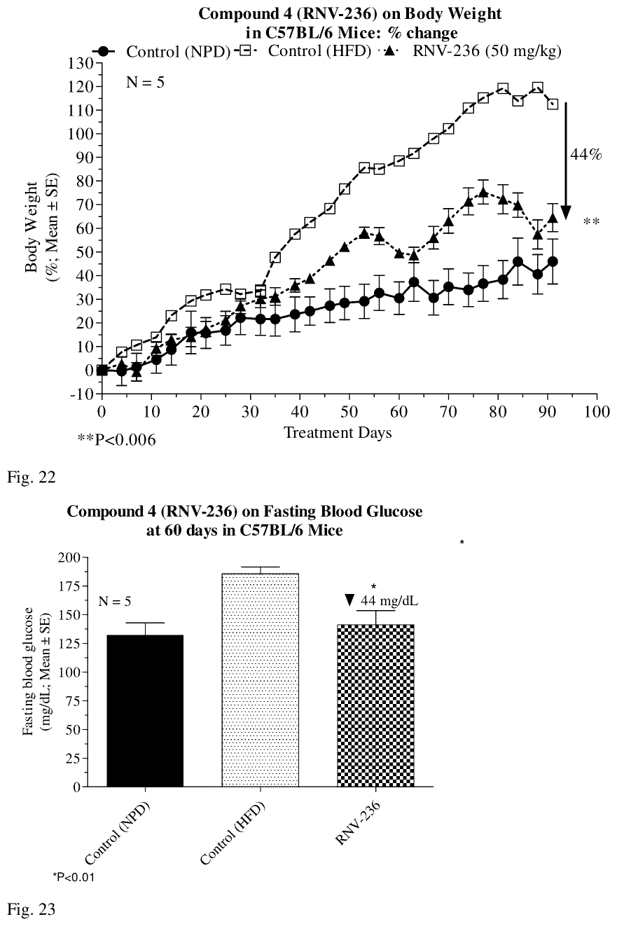

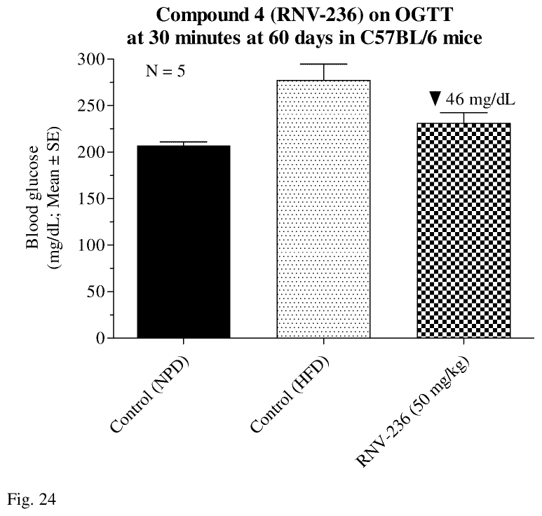

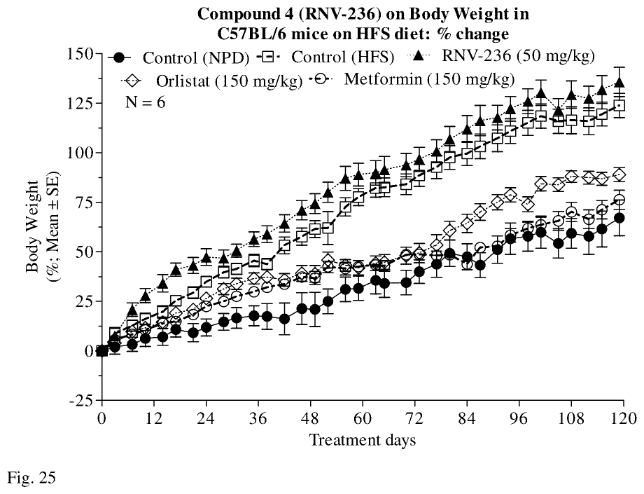

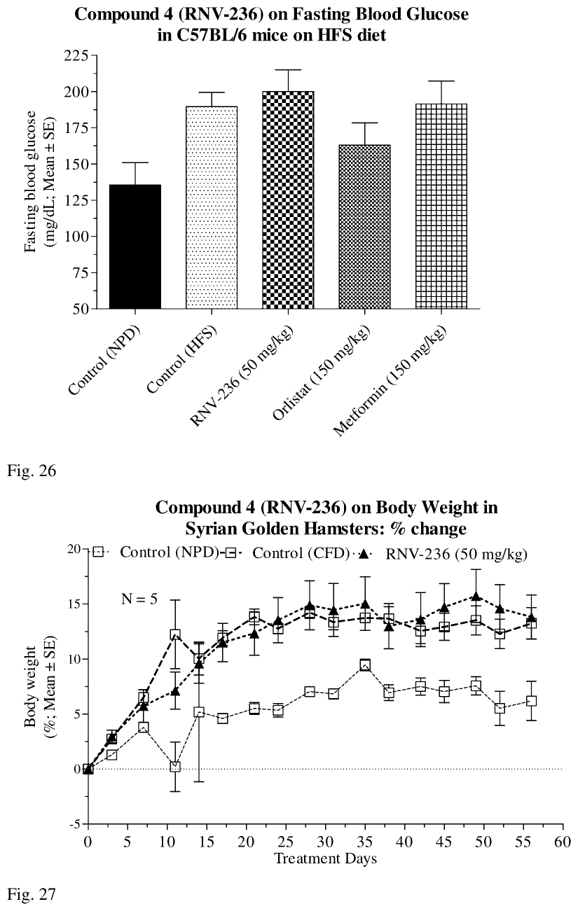

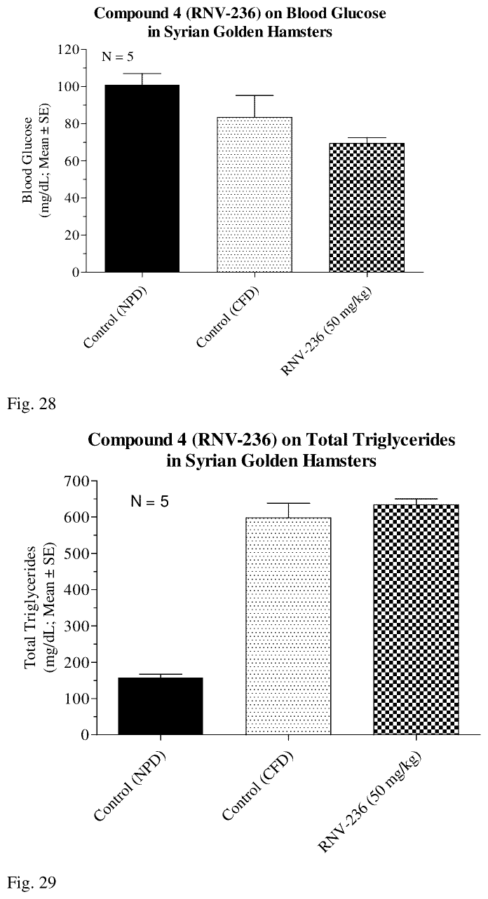

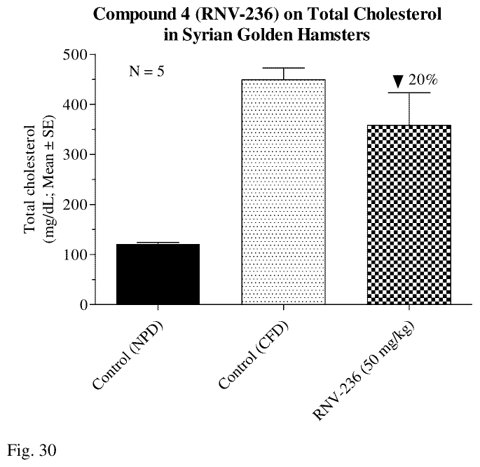

The formula of the useful compounds synthesized in this present are listed below. (See FIG. 3 for the R1, R2, R3 representations and IUPAC names for these compounds.)
Nicotinic acid 2-methoxy-4-[(1-methoxycarbonyl-2-methyl-propylimino)-methyl]-phenyl ester (RNV-133)
Nicotinic acid-4-(hydroxyimino-methyl)-2-methoxy-phenyl ester (RNV-168)
Nicotinic acid 4-[(2,4-dinitro-phenyl)-hydrazonomethyl]-2-methoxy-phenyl ester (RNV 203) Nicotinic acid 4-[(2,4-dinitro-phenyl)-hydrazonomethyl]-2-methoxy-6-Bromo phenyl ester (RNV-236)

Preferred salts for the compounds listed above are hydrochloride, hydrobromide, sodium, potassium or magnesium.

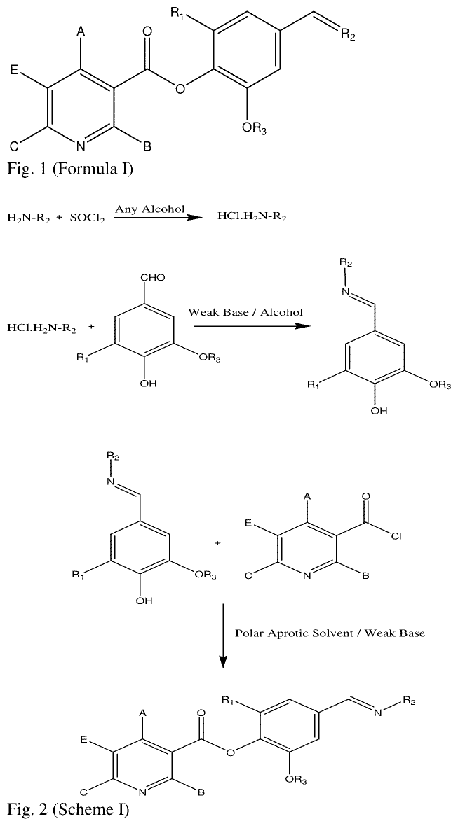

According to another feature of this present invention, there is provided a process for the preparation of the compound represented by the formula I, wherein all symbols are as defined as earlier, as shown in Scheme I (see FIG. 2).

Amino acids (naturally occurring and synthetic), their corresponding amino acid alcohols and amino acid esters, hydroxylamines and 2,4 or 3,5 di substituted phenyl hydrazines where the substituents can be H, Halogens or Nitro group, through their free amine group reacts with thionyl chloride in the presence of any alcohol to give a chloride salt of the corresponding amine. This salt reacts with substituted aromatic aldehyde in the presence of a weak base to give its corresponding Schiff base. This Schiff base reacts with terta substituted nicotonyl chloride in the presence of a weak base and using a polar apriotic solvent as the medium to yield Pyride Carboxylate derivatives of the general formula I wherein the substituents have been defined earlier.

Embodiments may include, for example: a compound of formula I (see FIG. 1). In the compound of formula I, the groups represented as A, B, C, and E are selected from hydrogen, hydroxyl, alkoxy, straight-chain or branched alkyl, and halogens such as chlorine, fluorine, bromine, and iodine. The group represented as R1 is selected from hydrogen, hydroxyl, alkoxy, straight-chain or branched alkyl, and halogens such as chlorine, fluorine, bromine, and iodine. The group represented as R3 is a methyl group. The group represented as R2 is selected from amino acids (naturally occurring and synthetic), corresponding amino acid alcohols and amino acid esters, hydroxylamines, and 2,4- or 3,5-disubstituted phenyl hydrazines, wherein the substituents are H, halogens, or a nitro group. Where R2 is an amino acid group, the amino acid is selected from alanine, glycine, arginine, asparagine, cysteine, glutamic acid, glutamine, histidine, isoleucine, leucine, lysine, methionine, ornithine, proline, serine, threonine, tryptophan, tyrosine, and the like, which may be unsubstituted or substituted, including derivatives such as esters and amides of the carboxylic acid, with preferred substituents including halogen, alkyl, alkoxy, aryl, heteroaryl, amino, and the like. The invention further includes pharmaceutically acceptable salts, including base addition salts such as alkali metal salts (Li, Na, K), alkaline earth metal salts (Ca, Mg), and salts of organic bases such as lysine, arginine, guanidine, diethanolamine, chlorine and the like, and ammonium or substituted ammonium salts, as well as acid addition salts including sulphates, nitrates, phosphates, perchlorates, borates, hydrohalides, acetates, tartarates, maleates, citrates, succinates, palmoates, methanesulphonates, benzoates, ascorbates, glycerophosphates, ketoglutarates, and the like; pharmaceutically acceptable solvates are also included, such as hydrates or solvates comprising other crystallization solvents including alcohols. The invention also includes derivatives, analogues, tautomeric forms, stereoisomers, polymorphs, pharmaceutically acceptable salts, pharmaceutically acceptable solvates, novel intermediates, and pharmaceutical compositions containing such compounds, wherein the pyridine moiety is unsubstituted (A=B=C=E=hydrogen) or substituted with hydroxyl, alkoxy, or halogen at suitable positions, the carboxylic group meta to the hetero nitrogen atom is esterified with the hydroxyl group of a vanillin moiety, and the aldehyde group of the vanillin moiety forms a condensation analogue with a free amino group of an alkyl or aryl amino acid, or with a hydroxylamine or hydrazine; in such embodiments, R1 is preferably selected from hydrogen, hydroxyl, alkoxy, straight-chain or branched alkyl, and halogens such as chlorine, fluorine, bromine, and iodine, R2 is preferably selected from amino acids (naturally occurring and synthetic), corresponding amino alcohols and amino esters, hydroxylamines, and 2,4- or 3,5-disubstituted phenyl hydrazines wherein substituents are H, halogens or nitro, and R3 is preferably selected from hydrogen or long- or short-chain alkyl groups that may be straight-chain or branched. Specific compounds include Nicotinic acid 2-methoxy-4-[(1-methoxycarbonyl-2-methyl-propylimino)-methyl]-phenyl ester (RNV 133), Nicotinic acid-4-(hydroxyimino-methyl)-2-methoxy-phenyl ester (RNV-168), Nicotinic acid 4-[(2,4-dinitro-phenyl)-hydrazonomethyl]-2-methoxy-phenyl ester (RNV 203), and Nicotinic acid 4-[(2,4-dinitro-phenyl)-hydrazonomethyl]-2-methoxy-6-bromo phenyl ester (RNV 236), with preferred salts including hydrochloride, hydrobromide, sodium, potassium, and magnesium salts. Also claimed are processes for preparing the novel compounds, including their analogues, tautomers, stereoisomers, polymorphs, pharmaceutically acceptable salts and solvates, novel intermediates, and pharmaceutical compositions containing them, wherein tautomeric forms are isomeric forms existing in equilibrium, stereoisomers include configurational isomers such as cis- and trans-double bonds and optically active isomers having different spatial atom arrangements, polymorphs are molecules capable of crystallizing in two or more forms, and solvates are molecular or ionic complexes of solvent molecules or ions with those of the solute; analogues include compounds differing by substitution of oxygen, sulphur, nitrogen or carbon atoms or substitution with atoms from the same periodic family such as F, Cl, Br and I, and derivatives include compounds resulting from routine functionalization such as protection of amino or carboxyl groups by carboxylation or esterification. A further claimed process for preparing the compound of formula I (see Scheme I, FIG. 2) comprises reacting amino acids (naturally occurring and synthetic), corresponding amino acid alcohols and amino acid esters, hydroxylamines, or 2,4- or 3,5-disubstituted phenyl hydrazines (with substituents H, halogens, or nitro) through their free amine group with thionyl chloride in the presence of an alcohol to form a chloride salt of the corresponding amine, reacting that salt with a substituted aromatic aldehyde in the presence of a weak base to yield a Schiff base, and reacting the Schiff base with a tetra substituted nicotinyl chloride in the presence of a weak base and in a polar aprotic solvent to yield pyridine carboxylate derivatives of the general formula I with substituents as defined above. Methods are also claimed for reducing plasma glucose, free fatty acids, cholesterol, and triglyceride levels, and for treating obesity, autoimmune diseases, inflammation, immunological disease, and disorders associated with insulin resistance, comprising administering to a patient in need thereof an effective amount of a compound of formula I as defined above; in particular, the autoimmune disease may be multiple sclerosis or rheumatoid arthritis, inflammation may be mediated by cyclooxygenase, and immunological disease may be mediated by cytokines.

1. A compound formula I 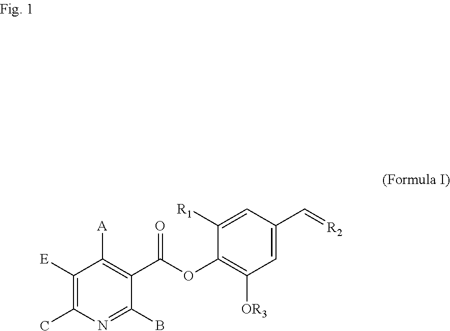
wherein, B, C, E are selected from Hydrogen, Hydroxyl, Alkoxy, alkyl groups with straight chain or branched and halogens like Chlorine, Fluorine, Bromine and Iodine; R1 can be selected from groups like Hydrogen, Hydroxyl, Alkoxy, alkyl groups with straight chain or branched and halogens like Chlorine, Fluorine, Bromine and Iodine; and R3 is a methyl group; and R2 can be selected from amino acids (naturally occurring and synthetic), their corresponding amino acid alcohols and amino acid esters, hydroxylamines and 2,4 or 3,5 disubstituted phenyl hydrazines where the substituents can be H, Halogens or Nitro group; and salts or derivatives thereof.

2. The compound of claim 1 selected from the group consisting of
Nicotinic acid 2-methoxy-4-[(1-methoxycarbonyl-2-methyl-propylimino)-methyl]-phenyl ester (RNV 133),
Nicotinic acid-4-(hydroxyimino-methyl)-2-methoxy-phenyl ester (RNV-168),
Nicotinic acid 4-[(2,4-dinitro-phenyl)-hydrazonomethyl]-2-methoxy-phenyl ester (RNV 203), and
Nicotinic acid 4-[(2,4-dinitro-phenyl)-hydrazonomethyl]-2-methoxy-6-Bromo phenyl ester (RNV 236).

3. The compound of claim 1 wherein R2 is selected from alanine, glycine, arginine, aspargine, cysteine, glutamic acid, glutamine, histidine, isoleucine, leucine, lysine, methionine, ornithine, proline, serine, threonine, tryptophan, tyrosine and the like, which may be unsubstituted or substituted and their derivatives such as ester and amides of carboxylic acid.

4. The compound of claim 1 wherein R2 is selected from a halogen, alkyl, alkoxy, aryl, heteroaryl, or amino group.

5. The compound of claim 1 wherein the salts are base addition salts.

6. The compound of claim 1 wherein the compound is a salt and is either a hydrochloride, hydrobromide salt, a sodium salt, a potassium salt or a magnesium salt.

7. A process for the preparation of the compound of claim 1 by the process shown:

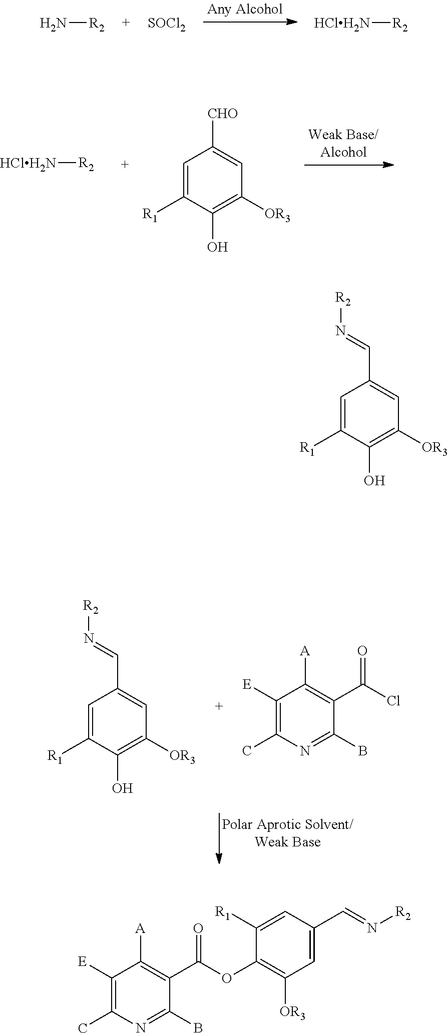

8. A method for treating a disease or affecting a physiological change in a subject comprising administering to the subject a pharmaceutically effective dose of the compound of claim 1, where the change comprises at least one change selected from the group consisting of: (i) reducing glucose in plasma; (ii) reducing free fatty acid in plasma; (iii) reducing cholesterol in plasma; and (iv) reducing triglyceride levels in plasma.

9. The method of claim 8 wherein the disease is selected from the group consisting of: obesity, an autoimmune disease, an inflammatory disease, and an immunological disease.

10. The method of claim 9 wherein the autoimmune disease is multiple sclerosis.

11. The method of claim 9 wherein the autoimmune disease is rheumatoid arthritis.

12. The method of claim 9 wherein the autoimmune disease is wherein the inflammatory disease is mediated by cyclooxygenase.

13. The method of claim 9 wherein the inflammatory disease wherein the immunological disease is mediated by cytokines.

14. The method of claim 8 wherein the disease is associated with insulin resistance.
